(19) United States
(12) Patent Application Publication
(10) Pub. No.: US 2011/0152710 A1
(43) Pub. Date: Jun. 23, 2011

(54) ADAPTIVE BRAIN-COMPUTER INTERFACE DEVICE

(75) Inventors: Kee-Eung KIM, Daejeon (KR); Jinhyung Kim, Seoul (KR); Jaeyoung Park, Uijeongbu-si (KR)

(73) Assignee: KOREA ADVANCED INSTITUTE OF SCIENCE AND TECHNOLOGY, Daejeon (KR)

(21) Appl. No.: 12/693,873

(22) Filed: Jan. 26, 2010

(30) Foreign Application Priority Data
Dec. 23, 2009 (KR) 10-2009-0129786

(51) Int. Cl. A61B 5/0478

(52) U.S. Cl. 600/545

(57) ABSTRACT

Disclosed herein is an adaptive brain-computer interface device. The brain-computer interface device includes a stimulus generation unit, a signal collection unit, a preprocessing unit, an analysis unit, and a stimuli sequence determination unit. The stimulus generation unit generates stimuli and applies the stimuli to a user. The signal collection unit records the user's Electroencephalogram (EEG) signals generated by the stimuli. The preprocessing unit extracts the feature of P300 on the basis of one of the stimulus from the EEG signals. The analysis unit determines whether P300 is present in the signal extracted by the preprocessing unit. The stimuli sequence determination unit determines the sequence of the stimuli of the stimulus generation unit by inferring a current state from the observations of the analysis unit and selecting an optimum stimulus for the current state.

### CROSS-REFERENCES TO RELATED APPLICATION

This patent application claims the benefit of priority under 35 U.S.C. §119 from Korean Patent Application No. 10-2009-0129786 filed on Dec. 23, 2009, the contents of which are incorporated herein by reference.

### BACKGROUND OF THE INVENTION

1. Field of the Invention

The present invention relates generally to an adaptive brain-computer interface device, and, more particularly, to an adaptive brain-computer interface device which is capable of increasing the number of instructions and messages which can be input per unit time.

2. Description of the Related Art

In general, a brain-computer interface refers to a device which issues instructions and messages to an external device using the brain's activities without requiring physical action. In particular, a brain-computer interface can be very helpful for handicapped persons expressing themselves and controlling devices.

In order to construct such brain-computer interfaces, an invasive method of directly inserting electrodes into a brain and measuring the activity of the brain and a noninvasive method of measuring the activity of a brain using brain waves observed outside the skull have been used.

An Electroencephalogram (EEG) is taken using the noninvasive method and is a record of electric signals which are generated by the neurons of a brain.

Such electric signals represent the sum of the simultaneous electrical activities of thousands or millions of neurons which are present around electrodes used to measure brain to waves.

When a somatic stimulus to which a user is paying attention is given, Event-Related Potentials (ERPs) based on the corresponding stimulus occur in measured brain waves.

P300 refers to a peak which is directed towards the positive (+) potential of ERPs which occur about 300 ms after a stimulus has been given. P300 is known to be the activity of a brain which can be reliably used to construct a brain-computer interface.

An example into which research has been actively conducted to construct a brain-computer interface using P300 is a P300 speller. A P300 speller is a device which enables a keyboard to be manipulated using brain waves.

In a P300 speller, characters are arranged in a 6×6 matrix, and a user fixes his or her eyes on one of the 36 characters which is desired to be input. The stimuli adapted such that the characters arranged in respective rows and columns are flashed during short periods of time in a random sequence are applied to the user.

If a stimulus related to a character on which a user fixes his or her eyes is applied to the user in the situation where the above-described stimuli are being given to the user, ERPs appear in brain waves and P300 will appear in brain waves about 300 ms after the above stimulus has been applied to the user.

The brain-computer interface enables such a P300 signal to be detected and a character on which a user fixes his or her eyes to be found. That is, if P300 is present in a brain signal corresponding to a specific stimulus, the user is interpreted as desiring to input a character corresponding to the corresponding stimulus. In contrast, if P300 is not detected, the user is interpreted as not desiring to input the character corresponding to the relevant stimulus.

Furthermore, it is possible to vary the number of available characters by adjusting the size of the matrix in the P300 speller. Moreover, it is possible to replace the characters with instructions and apply the brain-computer interface to the control of a device, such as a wheelchair.

FIG. 4 is a diagram showing a configuration of a conventional adaptive brain-computer interface device.

Meanwhile, the typical structure of the brain-computer interface device based on P300 is illustrated as shown in FIG. 4.

A stimulus generation unit 10 is a device for generating stimuli so that P300 can be generated under the conditions desired by a user. A signal collection unit 20 is a device for recording the user's brain waves for given stimuli. Furthermore, a preprocessing unit 30 performs preprocessing such as the extraction of P300 from a given brain wave signal. An analysis unit 40 functions to determine whether P300 is present in the extracted signal provided by preprocessing unit and to issue a command to an external device 50 connected to the brain-computer interface.

The above-described conventional brain-computer interface device applies the same number of stimuli for every type to a user so as to find an instruction desired to be input by the user, and the sequence of application of all types of stimuli is determined in a random manner.

However, the application of the same number of stimuli for every type is an unnecessary process, and the arbitrary determination of the sequence of application of stimuli limits improvements in the performance of a brain-computer interface.

For example, in the P300 speller, when the possibility of a user's intention being present in a first row is very low on the basis of stimuli given up to the present and EEG signals collected for them, there is no reason for applying a stimulus to the first row again.

Meanwhile, when the possibility of a user's intention being present on second and third rows is high, it will be better to reduce uncertainty by repeatedly applying a stimulus to the two rows.

That is, if the sequence of the application of stimuli to a user can be efficiently determined, the user's intention can be detected using only a small number of stimuli.

### SUMMARY OF THE INVENTION

Accordingly, the present invention has been made keeping in mind the above problems occurring in the prior art, and an object of the present invention is to provide an adaptive brain-computer interface device which is capable of adaptively determining the sequence of stimuli in accordance with a situation so that an instruction desired by a user can be accurately determined within a short period of time.

Another object of the present invention is to provide an adaptive brain-computer interface device which is capable of detecting a large number of instructions and messages per unit time and of being applied to the control of an external device even in the case of a handicapped person or in a situation when it is difficult to use the hands or feet.

In order to accomplish the above objects, the present invention provides an to adaptive brain-computer interface device, including a stimulus generation unit for generating stimuli and applying the stimuli to a user; a signal collection unit for recording the user's Electroencephalogram (EEG) signals generated by the stimuli; a preprocessing unit for extracting the feature of P300 on the basis of one of the stimulus from the EEG signals; an analysis unit for determining whether P300 is present in the signal extracted by the preprocessing unit; and a stimuli sequence determination unit for determining the sequence of the stimuli of the stimulus generation unit by inferring a current state from the observations of the analysis unit and selecting an optimum stimulus for the current state.

The stimuli sequence determination unit may determine an optimum action policy using a Partially Observable Markov Decision Process (POMDP).

The stimuli sequence determination unit may include a belief state update unit for inferring a distribution of probabilities of a current state from previous stimuli given to the user and observations corresponding to the respective stimuli; and an optimum stimulus selection unit for selecting and executing an optimum stimulus corresponding to a belief state of the belief state update unit, and transmitting an instruction or a message to an external device.

The stimuli sequence determination unit may determine an optimum action to policy using a delayed observation POMDP.

The stimuli sequence determination unit may determine an optimum action policy using only actions except for actions which had been taken for a period for which repetition blindness caused by repeated stimuli occurred.

The optimum action policy may be calculated using a value function defined by the following equation:

$$V * ( b ) = {max}_{A - {A}^{′}} [ R ( b , a ) + γ {\sum }_{z} P ( z \vert b , a ) V * ( τ ( b , a , z ) ) ]$$

when A is a set of actions and A′ is a set of actions performed within 500 ms from a reference time.

The preprocessing unit may remove noise by averaging the EEG signals or uses a P300 extraction algorithm such as a spatial filter algorithm or Mexican hat wavelet.

The analysis unit includes a P300 classifier using a classification algorithm such as Fisher's linear discriminant, a stepwise linear discriminant analysis, or a support vector machine.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a configuration of an adaptive brain-computer interface device according to an embodiment of the present invention;

FIG. 2 is a diagram showing another construction of an adaptive brain-computer interface device according to an embodiment of the present invention;

FIG. 3A is a graph showing results of experiments on the success rate in a 2×2 matrix;

FIG. 3B is a graph showing results of experiments on the success rate in a 2×3 matrix; and

FIG. 4 is a diagram showing a configuration of a conventional adaptive brain-computer interface device.

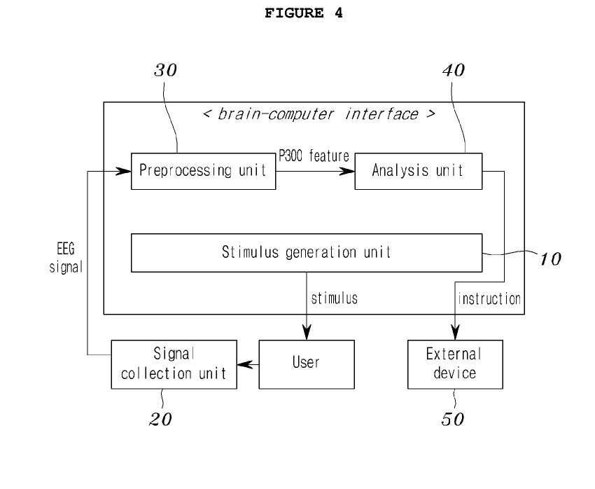

### DETAILED DESCRIPTION OF THE INVENTION

Reference now should be made to the drawings, in which the same reference numerals are used throughout the different drawings to designate the same or similar components.

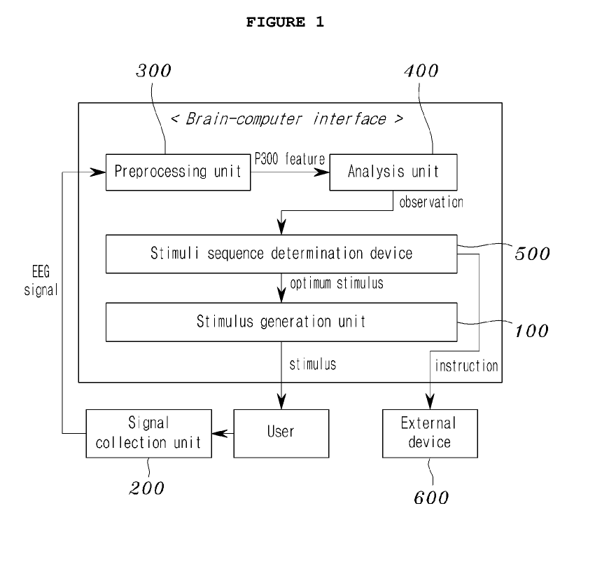

FIG. 1 is a diagram showing a configuration of an adaptive brain-computer interface device according to an embodiment of the present invention.

The adaptive brain-computer interface device according to the embodiment of the present invention, as shown in FIG. 1, includes a stimulus generation unit 100, a signal collection unit 200, a preprocessing unit 300, an analysis unit 400, and a stimuli sequence determination unit 500.

The stimulus generation unit 100 generates stimuli, and applies the stimuli to a user.

In detail, the stimulus generation unit 100 is a device which generates stimuli corresponding to respective instructions and applies the stimuli to a user so that P300, that is, the positive (+) direction peak of ERPs which is generated about 300 ms after a stimulus has been given, can be generated under a condition desired by the user.

A method by which the stimulus generation unit 100 applies stimuli to a user complies with a typical P300 speller system.

That is, a user fixes his or her eyes on a character desired to be selected, and to characters arranged in a matrix are flashed one at a time for 250 ms. In this case, for the first 125 ms of 250 ms, a character is brightened, and for the remaining 125 ms, the character is darkened.

When the task of finding a single character intended by a user is referred to as a test, an idle period of 2.5 seconds may be present between two successive tests.

The signal collection unit 200 is a device for recording a user's EEG signals generated by the stimuli.

The EEG signals may be collected from 16 channels at 1 kHz using a Biopac MP 150 system.

Since P300 is generated about 300 ms after a stimulus has been given, an EEG signal corresponding to a single flash of stimulus may be composed of data spanning from 200 ms after the stimulus has been given to 450 ms thereafter.

The preprocessing unit 300 is a device for extracting the feature of P300 based on stimulus from the EEG signals.

Since an EEG signal includes all of the brain activities which are generated due to a variety of causes, P300 does not directly appear because of the EEG signals generated due to other causes. That is, since the EEG signal includes a large amount of noise, it is difficult to to directly measure P300.

In order to solve this problem, a variety of machine learning techniques may be used.

The acquisition of refined brain waves using a filter so as to easily detect P300 is referred to as P300 feature extraction. For this purpose, noise can be removed by obtaining the average of brain waves or by using a P300 extraction algorithm, such as a spatial filter algorithm or Mexican hat wavelet.

The analysis unit 400 is a device for determining the presence of P300 in the extracted signal of the preprocessing unit 100.

The analysis unit 400 may be formed of P300 classifiers. The P300 classifiers may be a Fisher's linear discriminant, a stepwise linear discriminant analysis (SWLDA), and a Support Vector Machine (SVM).

In order to construct the preprocessing unit 300 and the classifier, training data, including EEG signals which are generated in a target case, that is, in the case where a stimulus is provided for a character on which a user fixes his or her eyes, and in a non-target case, that is, in the case when a stimulus is provided for a character on which the user does not fix his or her eyes, may be collected first.

Since the source EEG signals include a large amount of noise, the EEG signals may be subjected to band-pass filtering at 0.5˜30 Hz through a 6th order Butterworth filter, and may be down-sampled at 100 Hz.

A spatial projection algorithm may be used to extract features from the EEG signals collected as described above.

This algorithm calculates filters capable of converting EEG signals collected from respective channels into signals conceptually collected from a single virtual channel so as to maximally distinguish an EEG signal corresponding to a target from EEG signals corresponding to non-targets.

Meanwhile, in order to determine whether a feature extracted by the preprocessing unit 300 corresponds to a target in which P300 is present, a LIBLINEAR package may be used as the classifier, and in order to obtain a real number between 0˜1 instead of the binary output, an L2-regularized logistic regression may be used.

Here, the real number indicates the probability that a relevant feature corresponds to a target. The parameters of the classifier may be determined by 5-fold cross-validation based on the training data.

The stimuli sequence determination unit 500 is a device for determining the sequence of the stimuli of the stimulus generation unit 100 by inferring a current state from the observation by the analysis unit 400 and selecting an optimal stimulus for the current state.

In general, a Partially Observable Markov Decision Process (POMDP) is a general decision-making framework for dealing with a partially observable problem.

This is a model suitable for actual problems in which uncertainty is present, assumes an accurate mode for dealing with the actual world, and finds an optimal action policy for a given model.

The POMDP is defined by 8 elements <S,A,Z,b₀,T,O,R,γ>.

S is a state set, A is an action set, Z is an observation set, and bₒ is an initial belief state and b₀(s) is the probability of the state of an initial environment being ‘s’.

Furthermore, T is a state transition probability and T(s,a,s′) is the probability of transition from state s to state s′ by action a, and O is an observation probability and O(s,a,z) is the probability of reaching state s by action a and observing an observation z.

Furthermore, R is a compensation function and R(s,a) is a compensation obtained when action a is taken in state s, and γ is a discount rate and has the value of a real number between 0 and 1.

The agent of actions cannot directly detect the current environmental state, and can obtain only an observation instead. Accordingly, the agent of actions can maintain the distribution of probabilities of a current environmental state using all actions taken up to the present and observations adapted to correspond thereto. This is referred to as a belief state. bₜ(s) is the probability of the state being ‘s’ at time t.

When action aₜ is taken in the belief state at time t, that is, bₜ, and observation zₜ₊₁ is obtained, the belief state at the subsequent time, that is, bₜ₊₁=τ(bₜ,aₜ,zₜ₊₁), is calculated by the following Equation (1) on the basis of Bayes rule:

$${b}_{t + 1} ( {s}^{′} ) = \frac{O ( {s}^{′} , {a}_{t} , {z}_{t + 1} ) {\sum }_{s ∈ S} T ( s , {a}_{t} , {s}^{′} ) {b}_{t} ( s )}{P ( {z}_{t + 1} \vert {b}_{t} , {a}_{t} )} \qquad ( 1 )$$

where P(zₜ₊₁|bₜ,aₜ) is a normalization constant which enables

$${\sum }_{s} {b}_{t + 1} ( s ) = 1.$$

An action policy determines an action which is performed by the agent of actions, which is represented using a correspondence from a belief state to the action.

Every action policy has a corresponding value function, which represents the expected value of a discounted compensation function which can be obtained when actions are infinitely performed at a given belief state in compliance with the corresponding action policy.

Accordingly, solving a POMDP means calculating an action policy which enables the greatest value to be obtained in each belief state.

The greatest value obtainable at a specific belief state is recursively calculated using the following Equation (2):

$$V * ( b ) = {max}_{a} [ R ( b , a ) + γ {\sum }_{z} P ( z \vert b , a ) V * ( τ ( b , a , z ) ) ] \\ P ( z \vert b , a ) = {\sum }_{{s}^{′}} O ( {s}^{′} , a , z ) {\sum }_{s} T ( s , a , {s}^{′} ) b ( s ) \\ R ( b , a ) = {\sum }_{s} R ( s , a ) b ( s ) \qquad ( 2 )$$

Furthermore, the optimum action policy corresponding to the given optimum value function is calculated using the following Equation (3):

$$π * ( b ) = {arg max}_{a} [ R ( b , a ) + γ {\sum }_{z} P ( z \vert b , a ) V * ( τ ( b , a , z ) ) ] \qquad ( 3 )$$

Since it is impossible to actually calculate the optimum action policy because it takes an excessively long time, one of the optimum action policy approximation algorithms, such as point-based value iteration, may be used.

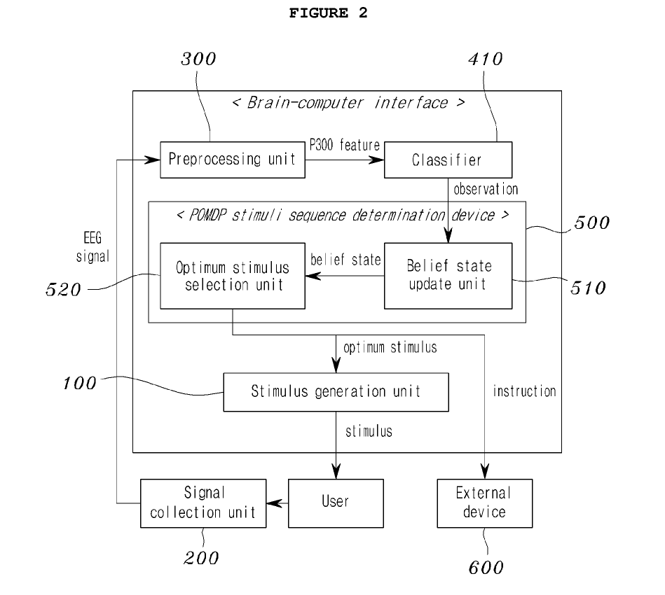

FIG. 2 is a diagram showing the construction of an adaptive brain-computer interface device according to an embodiment of the present invention.

The stimuli sequence determination unit 500 can determine the optimum action policy using a POMDP.

In this case, the given environment of a brain-computer interface must be modeled using the POMDP, and the optimum action policy must be calculated from the given POMDP model. Thereafter, the belief state update unit 510 and the optimum stimulus selection unit 520 may be constructed using the POMDP model and the optimum action policy of the corresponding POMDP model, as shown in FIG. 2.

The optimum action policy is the same as the optimum sequence of actions in a brain-computer interface, and determines the sequence of stimuli which enables an instruction desired by a user to be found with the highest accuracy within the shortest period of time in the situation in which a large amount of noise is included in observations.

The belief state update unit 510 may infer the distribution of the probabilities of a current state from previous stimuli given to a user and observations corresponding to the respective stimuli by using a belief state update method in a general POMDP.

That is, the belief state update unit 510 is a device for inferring the distribution of probabilities regarding how each instruction is currently desired to be input by a user.

The optimum stimulus selection unit 520 selects and processes the optimum stimulus corresponding to the belief state of the belief state update unit 510, and may transmit an instruction or message to an external device 600.

That is, the optimum stimulus selection unit 520 selects and performs an action corresponding to a current belief state using the previously calculated optimum action policy or approximation optimum action policy.

When the environment of the brain-computer interface is modeled using a POMDP as described above, a corresponding model enables the optimum action policy, capable of accurately finding an instruction desired by a user using the minimum number of stimuli, to be acquired.

This is very similar to the tiger problem in the POMDP field. This is thought of as having the form in which the number of queries in the tiger problem is extended to the number of instructions in the brain-computer interface.

Assuming that N is the number of instructions, respective instructions may be constructed using respective states (N states) in the POMDP, giving stimuli corresponding to respective instructions to a user or selecting respective instructions may be constructed using actions (2*N actions), and real numbers output from the classifier may be constructed using successive observations or may be constructed by discretizing them and then constructing them using respective observations (K observations).

Furthermore, a low compensation may be set for an action of giving a stimulus, a high compensation may be set for the case where the action of selection an instruction on which a user fixes his or her eyes is taken, and a very low compensation may be set for the case when the action of selection an instruction on which a user does not fix his or her eyes.

Furthermore, with regard to the state transition function, for the action of giving a stimulus, the state is the same as the previous state, and, for the action of selecting an instruction intended by a user, state transition occurs at the same probability for every character.

Furthermore, since in the case of an observation function, a large amount of noise is included in brain waves, it is impossible to exactly model an actual environment in which a brain-computer interface operates. However, data is collected from an existing noninvasive brain-computer interface, to which a POMDP has not been applied, through previous experiments, and then the data is used by fitting it into a beta distribution, an exponential distribution or an arbitrary probability distribution.

Moreover, the discount rate can be adjusted to a real number between 0 and 1 so that the agent of actions can take an appropriate action. The initial belief state is a probability distribution, and may be appropriately adjusted in accordance with an initial environmental state.

Meanwhile, a state in the POMDP may be thought to be an instruction currently desired to be selected by a user, whereas the brain-computer interface cannot directly detect this state.

Accordingly, an instruction desired to be input by a user must be inferred from stimuli given to the user and observations adapted to correspond to respective stimuli on the basis of the distribution of probabilities of respective states, that is, belief states.

If the brain-computer interface applies a single stimulus to a user and the corresponding stimulus is a stimulus related to an instruction desired to be input by a user, the probability of a given observation being an observation which frequently occurs when the stimulus related to the instruction desired to be input by the user is applied to the user is increased.

In contrast, when a given stimulus is not a stimulus related to an instruction desired to be input by the user, the probability of a given observation not being an observation which frequently occurs when the stimulus related to the instruction desired to be input by the user is applied to the user is increased.

That is, from an action of giving a single stimulus and a corresponding observation value, the probability of an instruction corresponding to each stimulus being an instruction desired by a user may be inferred on the basis of the belief state in the POMDP. When the probability of a specific instruction being an instruction desired by a user is relatively high, the brain-computer interface further generates a stimulus related to the corresponding instruction, thus enabling the determination of whether the corresponding instruction is the instruction desired by the user.

Thereafter, when the probability of a specific instruction being an instruction desired by a user is increased to a level equal to or higher than a predetermined level, the action of maximizing the expected value of a value which can be acquired from a given POMDP model is taken by selecting the corresponding instruction, rather than taking the action of giving a stimulus.

Meanwhile, calculating the optimum action policy from the above-described POMDP model may be performed using existing optimum action policy calculation algorithms.

However, in general, the problem of determining the optimum action policy of a POMDP is known as PSPACE-Complete. For problems including large numbers of states, actions and observations, it takes an excessively long time to calculate an accurate action policy, so that actual calculation is impossible. In order to overcome this, an optimum action policy approximation algorithm, such as point-based value iteration or heuristic search value iteration, may be employed.

Using this approximation algorithm, the optimum action policy approximated to a POMDP model of appropriate size can be sufficiently calculated.

Meanwhile, the stimuli sequence determination unit 500 may determine the optimum action policy using a delayed observation POMDP.

Although in a basic POMDP, it may be assumed that after one action has been performed, a relevant observation is given before a subsequent action is performed, this assumption need not be true in an actual brain-computer interface.

For example, when stimuli are applied to a user at intervals of 250 ms, the length of brain waves to be used as an observation corresponding to a first stimulus may exceed 250 ms. In particular, in a brain-computer interface using P300, P300 can be detected 300 ms after a stimulus has been applied to a user, so that an observation related to a first stimulus can be acquired at least after a second stimulus has been given.

This is referred to as a delayed observation. Although this restriction does not exert an influence on an actual POMDP modeling process at all, a problem may occur in a process of calculating the optimum action policy of a corresponding POMDP model. This restriction may be overcome using a POMDP with delayed observations.

Furthermore, the stimuli sequence determination unit 500 may determine the optimum action policy using all actions except for actions which had been taken during a period for which repetition blindness caused by repeated stimuli occurred.

Repetition blindness caused by repeated stimuli refers to a phenomenon in which, for example, when it is assumed that an instruction intended by a user is “A” and a stimulus corresponding to “A” is applied to the user again within 500 ms after a stimulus corresponding to “A” has been applied to the user, P300 does not occur in brain waves related to the second stimulus.

A simple method of solving the above problem is not to apply to a user a stimulus which was applied to the user within 500 ms before the repeated application of the stimulus.

Although this does not exert an influence on actual POMDP modeling, a problem occurs in a process of calculating the optimum action policy of a corresponding POMDP model. A method of solving the above problem may be calculated by, assuming that A is a set of actions and A′ is a set of actions which have been taken within 500 ms before a reference time, defining a value function, as expressed in the following Equation 4:

$$V * ( b ) = {max}_{A - {A}^{′}} [ R ( b , a ) + γ {\sum }_{z} P ( z \vert b , a ) V * ( τ ( b , a , z ) ) ] \qquad ( 4 )$$

An experimental example using the adaptive brain-computer interface device according to an embodiment of the present invention will be described below.

In the following experimental example, nine persons having no physical and psychological abnormalities participated in the experiment. A baseline case is the same as the present invention except that the sequence of application of stimulus is arbitrary, whereas a PWSA refers to a device to which a POMDP has been applied.

In order to compare the Baseline case with the PWSA case in a fair manner, the baseline case was executed in such a way that a stimulus was randomly selected from among stimuli other than two previous stimuli, thereby overcoming repetition blindness.

### Experimental Example

First, 9 POMDP models having different observation probabilities were created for a 2×2 matrix and 11 different models were created for a 2×3 matrix, and the action policies of the corresponding models were calculated prior to the experiment.

In the present experiment, in order to create a preprocessing unit and a classifier for each person in the experiment, training data was collected first.

Thereafter, baseline and PWSA experiments were carried out on each of the 2×2 and 2×3 matrices. In the PWSA case, a POMDP model most similar to the distribution of observations for the corresponding person in the experiment was searched for, and the sequence of stimuli was determined using the action policy of the corresponding model.

The baseline and PWSA cases were compared with each other by using success rate and bit rate as evaluation factors. Here, success rate is defined as the ratio of the number of hits at a user's intention to the number of given stimuli, and bit rate represents the amount of information transmitted per unit time.

Although actually 9 persons participated in the experiment, the observational probabilities for 2 persons were very different from those of the previously created POMDP models, so that data related to the 2 person was excluded form experiment results.

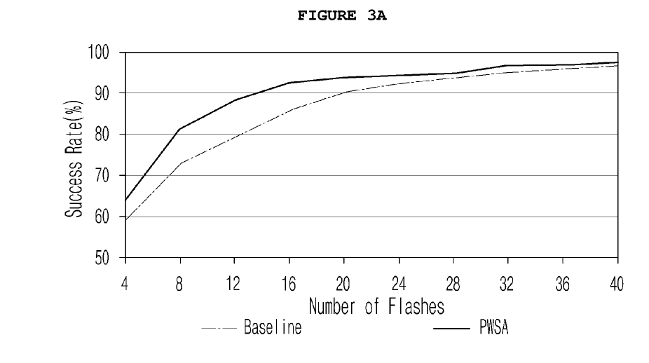

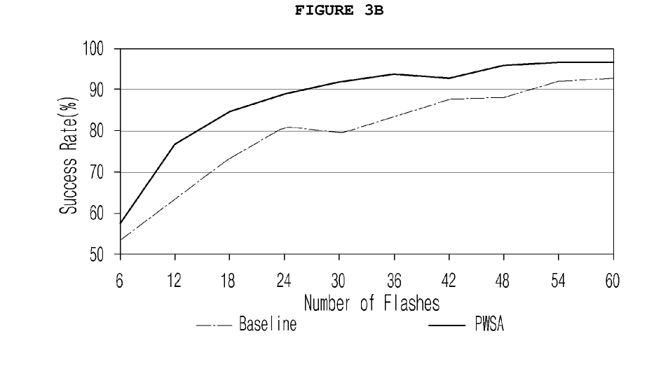

FIG. 3A is a graph showing results of experiments on the success rate in a 2×2 matrix, and FIG. 3B is a graph showing results of experiments on the success rate in a 2×3 matrix.

From FIGS. 3A and 3B, it can be seen that the success probability of the PWSA case was higher than that of the baseline case with respect to an arbitrary number of stimuli and the difference in the success rate is increased in proportion to the size of the matrix. Furthermore, it can be seen that in the PWSA case, convergence into a high success rate is achieved at a faster speed.

Meanwhile, results of experiments on the bit rate are listed in the following table 1:

TABLE 1

| | 2 × 2 Matrix | 2 × 3 Matrix |
|---|---|---|
| Baseline | 10.065 (96.4%) | 8.052 (92.9%) |
| PWSA | 24.368 (98.2%) | 21.367 (97.6%) |

As listed in Table 1, in the case of the 2×2 matrix, the PWSA case exhibited a bit rate of 24.368 bits/min at a success rate of 98.2%. In the case of the 2×3 matrix, the PWAS case exhibited a bit rate of 21.367 bits/min at a success rate of 97.6%.

For the respective matrices, the baseline case exhibited maximum success rates of 96.4% and 92.9%, and the bit rates for the respective success rates were merely 10.065 bits/min and 8.052 bits/min.

The adaptive brain-computer interface device according to the present invention has the advantage of adaptively determining the sequence of stimuli in accordance with a situation so that an instruction desired by a user can be accurately found within a short period of time.

The adaptive brain-computer interface device according to the present invention has the advantage of detecting a large number of instructions and messages per unit time and being applied to the control of an external device even in the case of a handicapped person or in the situation where it is difficult to use the hands and feet.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An adaptive brain-computer interface device, comprising:
a stimulus generation unit for generating stimuli and applying the stimuli to a user;
a signal collection unit for recording the user's Electroencephalogram (EEG) signals generated by the stimuli;
a preprocessing unit for extracting a feature of P300 based on one of the stimulus from the EEG signals;
an analysis unit for determining whether P300 is present in the signal extracted by the preprocessing unit; and
a stimuli sequence determination unit for determining a sequence of the stimuli of the stimulus generation unit by inferring a current state from observations of the analysis unit and selecting an optimum stimulus for the current state.

2. The adaptive brain-computer interface device as set forth in claim 1, wherein the stimuli sequence determination unit determines an optimum action policy using a Partially Observable Markov Decision Process (POMDP).

3. The adaptive brain-computer interface device as set forth in claim 2, wherein the stimuli sequence determination unit comprises:
a belief state update unit for inferring a distribution of probabilities of a current state from previous stimuli given to the user and observations corresponding to the respective stimuli; and
an optimum stimulus selection unit for selecting and executing an optimum stimulus corresponding to a belief state of the belief state update unit, and transmitting an instruction or a message to an external device.

4. The adaptive brain-computer interface device as set forth in claim 1, wherein the stimuli sequence determination unit determines an optimum action policy using a delayed observation POMDP.

5. The adaptive brain-computer interface device as set forth in claim 1, wherein the stimuli sequence determination unit determines an optimum action policy using only actions except for actions which had been taken for a period for which repetition blindness caused by repeated stimuli occurred.

6. The adaptive brain-computer interface device as set forth in claim 5, wherein the optimum action policy is calculated using a value function defined by the following equation:
$$V * ( b ) = {max}_{A - {A}^{′}} [ R ( b , a ) + γ {\sum }_{z} P ( z \vert b , a ) V * ( τ ( b , a , z ) ) ]$$
where A is a set of actions and A′ is a set of actions performed within 500 ms from a reference time.

7. The adaptive brain-computer interface device as set forth in claim 1, wherein the preprocessing unit removes noise by averaging the EEG signals or uses a P300 extraction algorithm such as a spatial filter algorithm or Mexican hat wavelet.

8. The adaptive brain-computer interface device as set forth in claim 1, wherein the analysis unit comprises a P300 classifier using a classification algorithm such as Fisher's linear discriminant, a stepwise linear discriminant analysis, or a support vector machine.
